SEQUENCE LISTING (5 sequences)

SEQ ID NO: 1 - DNA, 297 bp, Homo sapiens
  CDS (1)..(297)
gcaatcgatggggcatcctttctgaagatcttcgggccactgtcgtccagtgccatgcagtttgtcaacgtgggctacttcctcatcgcagccggcgttgtggtctttgctcttggtttcctgggctgctatggtgctaagactgagagcaagtgtgccctcgtgacgttcttcttcatcctcctcctcatcttcattgctgaggttgcagctgctgtggtcgccttggtgtacaccataatggctgagcacttcccgacgttgctggtagtgcctgccatcaagaagattatggtt

SEQ ID NO: 2 - protein, 99 aa, Homo sapiens
AIDGASFLKIFGPLSSSAMQFVNVGYFLIAAGVVVFALGFLGCYGAKTESKCALVTFFFILLLIFIAEVAAAVVALVYTIMAEHFPTLLVVPAIKKIMV

SEQ ID NO: 3 - DNA, 261 bp, Homo sapiens
agccagcgaacggacgagggtgacaatagagtgtggtgtcatgcttgtgagagagaaaacactttcgagtgccagaacccaaggaggtgcaaatggacagagccatactgcgttatagcggccgtgaaaatatttccacgttttttcatggttcgcaacaggtgctccgctggttgtgcagcgatggagagacccaagccagaggagaagcggtttctcctggaagagcccatgcccttcttttacctcaagtgttgtaaa

SEQ ID NO: 4 - DNA, 10 bp, Homo sapiens
agccagcgaa

SEQ ID NO: 5 - DNA, 10 bp, Homo sapiens
gcaatcgatg

(19) United States
(12) Patent Application Publication
(10) Pub. No.: US 2008/0176214 A1
(43) Pub. Date: Jul. 24, 2008

(54) DNA for evaluating the progression potential of cervical lesions

(76) Inventors: Matthias Durst, Heidelberg (DE); Matthias Nees, Rockville, MD (US)

Correspondence Address: MOORE & VAN ALLEN PLLC, P.O. BOX 13706, Research Triangle Park, NC, 27709, US

(21) Appl. No.: 11/545,970

(22) Filed: Oct. 10, 2006

Related U.S. Application Data
(63) Continuation of application No. 10/079,954, filed on Feb. 19, 2002, now abandoned.
Application No. 10/079,954 is a continuation of application No. 09/308,984, filed on Sep. 3, 1999, now Pat. No. 6,388,065.

(51) Int. Cl. C12Q 1/70; C07K 16/18

(52) U.S. Cl. 435/5; 530/387.9

(57) ABSTRACT

The present invention relates to an nucleic acid which is suitable for evaluating the progression potential of cervical lesions, wherein the nucleic acid is obtainable by a process in which RNA from early and late passages of HPV-immortalized cells is isolated and the RNAs characteristic for the early passages and late passages, respectively, are identified and provided as DNA or RNA. Furthermore, the present invention concerns polypeptides coded by such a nucleic acid. In addition, it covers antibodies directed against the polypeptides. Moreover, it relates to the use of the DNA and the polypeptides as well as a kit suitable for evaluating the progression of cervical lesions.

### RELATED APPLICATION

This application is a of application Ser. No. 10/079,954, filed Feb. 19, 2002, which is a continuation of application Ser. No. 09/308,984, filed Sep. 3, 1999, now U.S. Pat. No. 6,388,065, incorporated by reference in their entireties.

This invention relates to nucleic acid molecules suitable for evaluating the progression potential of cervical lesions, and to polypeptides encoded by such a nucleic acid molecules. Furthermore, this invention concerns antibodies directed against the polypeptides. Moreover, it covers the use of the nucleic acid molecules and the polypeptides as well as a kit suitable for evaluating the progression of cervical lesions.

Invasive cervical carcinoma usually follows from a precancerosis. Precancerosis cover a wide range of lesions referred to as mild to severe dysplasias (CIN1 to CIN3) pertaining to histopathology. CIN1 lesions frequently regress spontaneously and usually need not be treated. On the other hand, these lesions can persist over years or change into a more severe lesion, e.g. CIN3, or into a microinvasive carcinoma. A cytological method has been used for the diagnosis of cervical smears for about 50 years, by means of which dysplastic cells can be detected in cervical smears. This method is generally known as the “Pap test”. The “Pap test” contributed to the fact that the incidence of the cervical carcinoma could be reduced significantly in the past.

Several years ago, it was also found that the presence of dysplastic lesions and cervical carcinomas correlates with the detection of cervical carcinoma-associated human papilloma viruses, e.g. HPV 16 or HPV 18. The detection of antibodies against the viral HPV oncoproteins E6 and E7 in patient serum by means of ELISA or comparable methods is also possible as a detection of premalignant or malignant cervical diseases. It is also known that certain chromosomal deletions are associated with an increased risk of malignant transformation of the corresponding precancerosis. Morphological changes of cells and cell nuclei also correlate with malignant progression. These changes can be detected by means of cytometry.

Nevertheless, it is not possible by either the “Pap test” or the detection of oncogenic HPV types to make a prognosis regarding the further development of individual lesions. This also applies to the detection of HPV-specific antibodies in patient serum. By means of this serological method it is rather only possible to detect patients who already suffer from a carcinoma. Therefore, the antibody detection method cannot be considered a supplement to the present precaution but only to the manifestation of the finding that a carcinoma had developed already. In addition, the genetical analysis and cytometric approaches include the drawback that they are technically very complicated and therefore are not useful for routine diagnostics.

Therefore, it is the object of the present invention to provide a product by which the progressive potential of cervical lesions can be evaluated reliably.

This is the subject of the invention set forth in the disclosure which follows.

Thus, the subject matter of the present invention relates to an isolated nucleic acid molecule suitable for evaluating the progressive potential of cervical lesions. Such a nucleic acid molecule can be obtained by common methods. A method is preferred in which RNA from early and late passages of HPV-immortalized cells is isolated and the RNAs characteristic of the early passages and late passages, respectively, and expressed in markedly differing amounts, respectively, are identified and provided as DNA or RNA.

The present invention is based on the applicant's finding that late passages of HPV-immortalized cells cause tumors in naked mice, whereas early passages of such cells are not capable of doing so. The inventor also discovered that in late passages of HPV-immortalized cells certain RNAs are detected more strongly than is the case in early passages of such cells.

For the provision of a nucleic acid according to the invention RNA from early and late passages of HPV-immortalized cells are isolated. Early passages are e.g. passage 20-60, and late passages start from e.g. 130. For example, HPV 16-immortalized, human preputial keratinocytes, HPK-IA cells can be used as cells (cf. Dürst, M. et al., Oncogene 1/3 (1987), 251-256). The RNA of the early and late passages can be compared with each other and differences can be determined which are characteristic of the early passages and late passages, respectively. To this end, it is favorable to subject the RNA to reverse transcription. In this case, it is advantageous to use what are known as anchor primers, i.e. oligo-d(T) primers which following a sequence of 11-15 thymidine bases have two more bases at the 3′ end and thus recognize in well-known fashion the transition from the 3′ end of an mRNA to the poly-A tail where they bind. The resulting cDNA can be subjected to amplification in a PCR method. For this purpose, it is favorable to use common “arbitrary” primers together with the above anchor primers. The amplified cDNA can then be subjected to denatured polyacrylamide gel electrophoresis. In this step, cDNA bands are identified which have a differing intensity in the cDNA samples to be compared with one another, i.e. RNA isolates from the early and late passages of HPV-immortalized cells. These cDNA bands can be isolated from the gel and subjected to another round of amplification. Moreover, they can be cloned and their sequences can be determined. A person skilled in the art is familiar with the above methods. By way of supplement reference is made to the following literature (cf. Liang et al., Cancer Research 5, (1992), 6966-6968; Liang et al., Science 257, (1992), 967-971; Liang et al., Nucleic Acids Research 21, (1993), 3269-3275).

Both cDNA and RNA are examples of nucleic acid molecules in accordance with the invention, with cDNA being preferred. A (c)DNA which comprises a base sequence of FIG. 1 of FIG. 2 or a sequence differing therefrom by one or several base pairs is particularly preferred. The (c)DNA of FIG. 1 was deposited as C4.8 with the DSMZ (Deutsche Sammlung von Miroorganismiien und Zellkulturen) {Germany-type collection of microorganisms and cell cultures} under DSM 11197 on Oct. 4, 1996. Furthermore, the (c)DNA of FIG. 2 was deposited as C21.7 with the DSMZ under DSM 11198 on Oct. 4, 1996. A nucleic acid molecule according to the invention is described as DNA by way of example below.

A DNA according to the invention can be present in a vector, an expression vector, respectively. The person skilled in the art is familiar with examples thereof. In the case of an expression vector for E. coli these include e.g. pGEMEX, pUC derivatives, pGEX-2T, pQE-8 and pet3d. For the expression in yeast e.g. pY100 and Ycpad1 have to be mentioned, while e.g. pKCR, pEFBOS, cDM8 and pCEV4 have to be indicated for the expression in animal cells. The baculovirus expression vector pAcSGHisNT-A is particularly suitable for the expression in insect cells.

The person skilled in the art knows suitable cells which express DNA according to the invention, which is present in an expression vector. Examples of such cells comprise the E. coli strains HB101, DH, x1776, JM101, JM109, SG13009 and BL21, the yeast strain Saccharomyces cerevisiae and the animal cells L, 3T3, FM3A, CHO, COS, Vero and HeLa as well as the insect cells sf9.

The person skilled in the art knows in which way a DNA according to the invention has to be inserted in an expression vector. He is also familiar with the fact that this DNA can be inserted in combination with a DNA coding for another polypeptide, so that the DNA according to the invention can be expressed in the form of a fusion protein.

In addition, the person skilled in the art knows conditions of culturing transformed cells and transfected cells, respectively. He is also familiar with methods of isolating and purifying the polypeptide expressed by the DNA according to the invention. Thus, such a polypeptide, which may also be a fusion polypeptide, also represents a feature of the present invention. A polypeptide in accordance with the invention comprises the amino acid sequence of FIG. 1 or a sequence differing therefrom by one or several amino acids.

A further aspect of the present invention relates to an antibody directed against an above polypeptide and fusion polypeptide, respectively. Such an antibody can be prepared by common methods. It may be polyclonal and monoclonal, respectively. For its preparation it is favorable to immunize animals—particularly rabbits or chickens for a polyclonal antibody and mice for a monoclonal antibody—with an above (fusion) polypeptide. Further “boosters” of the animals can be effected with the same (fusion) polypeptide. The polyclonal antibody can then be obtained from the animal serum and egg yolk, respectively. For the preparation of the monoclonal antibody, animal spleen cells are fused with myeloma cells.

The present invention enables the reliable evaluation of the progressive potential of cervical lesions. By means of an antibody according to the invention it can be determined whether cervical smears contain polypeptides which are characteristic of early or late passages of HPV-immortalized cells. Furthermore, it is possible to detect an autoantibody directed against the polypeptide present in the body by means of a polypeptide according to the invention. Both detections can be made by common methods, particularly a Western blot, an ELISA, an immunoprecipitation or by immunofluorescence. In addition, it is possible by means of a nucleic acid molecule according to the invention, particularly DNA and primers derived therefrom, to detect whether RNA which is characteristic of early or late passages of HPV-immortalized cells is present in cervical smears. This detection can be made in accordance with known methods, particularly, in a Southern blot. By means of the present invention it is thus possible, to make an early diagnosis of whether a cervical carcinoma is forming.

Furthermore, the present invention is useful for preventing the formation of a cervical carcinoma. By means of an antibody according to the invention it is possible to inhibit a polypeptide which is characteristic of late passages of HPV-immortalized cells. Moreover, a nucleic acid molecule according to the invention, particularly DNA, can be used to inhibit such a polypeptide. To this end, the nucleic acid molecule is used for the inhibition of expression of the gene coding for the polypeptide, e.g. as a basis of preparing anti-sense oligonucleotides.

To carry out the invention, particularly as regards the diagnostic aspect, a kit is also provided. It contains one or several nucleic acid molecules, polypeptides and/or antibodies according to the invention. In particular, it comprises those nucleic acid molecules and/or polypeptides which are said to be preferred above. In addition, the kit contains conventional excipients such as carriers, buffers, solvents and controls. The kit is also the subject matter of the present invention.

The present invention is explained by the examples which follow.

### BRIEF DESCRIPTION OF THE DRAWINGS

FIG. 1 shows the base sequence of (c)DNA C4.8 according to the invention. In addition, the amino acid sequence of the polypeptide encoded by (c)DNA C4.8 is indicated,

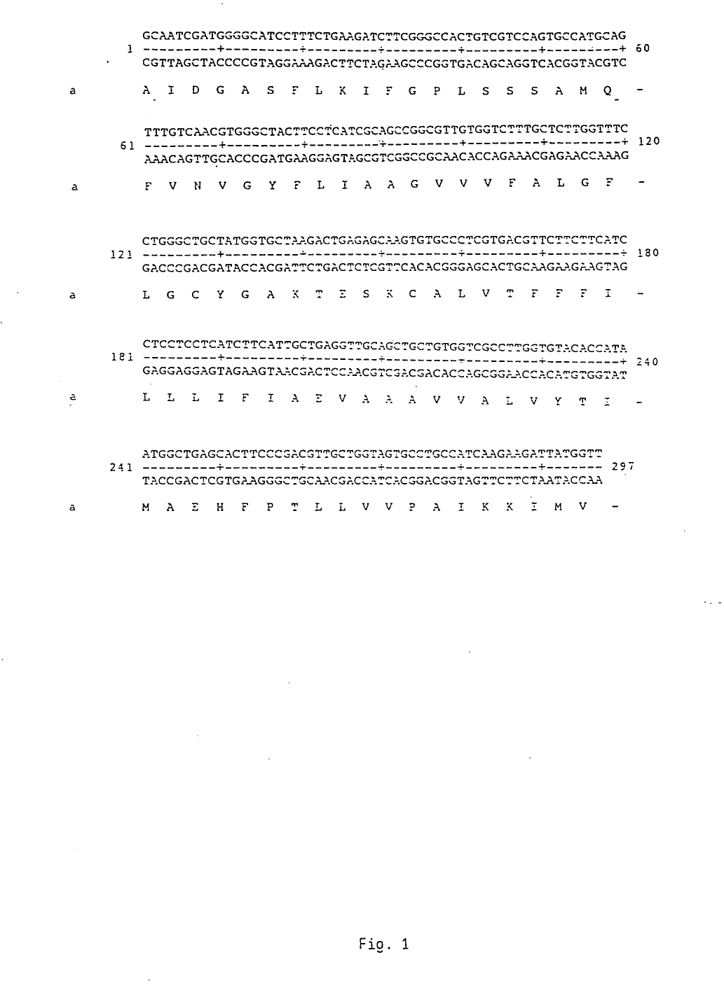

FIG. 2 shows the base sequence of (c)DNA C21.7 according to the invention.

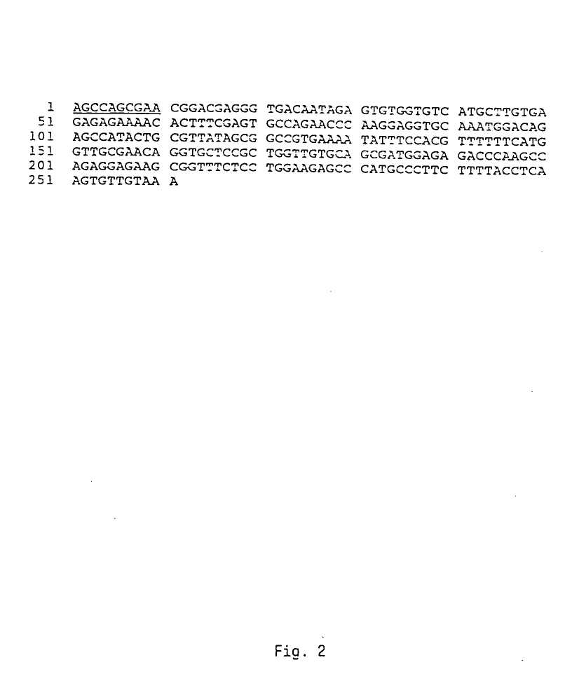

### EXAMPLE 1

### Preparation of cDNAs C4.8 and C21.7 According to the Invention

Whole RNA was isolated in each case by the known guanidine thiocyanate (GTC) method from early passages, i.e. passage 49, and late passages, i.e. passage 359 and p389, of the HPV-immortalized cell line HPK-IA. The whole RNA was subjected to a conventional DNase reaction, using commercially available RNase-free DNase purchased from PROMEGA.

The resulting DNase-free whole RNA was subjected to reverse transcription using anchor primers, as are described supra. Following a sequence of 11-15 thymidine bases, these primers have two more bases at the 3 end, e.g. AA, AC, AG, CA, CC, CG, GA, GC, GG, AT, CT, GT, so that the primers are bound directly at the transition from the mRNA to the poly-A tail.

The conditions for reverse transcription were as follows:

| | |
|---|---|
| RNasine 20 u/μl | 1.0 μl |
| dNTP-Mix (2.5 mM) | 1.2 μl |
| MMLV reverse transcriptase 300 u/μl | 2.5 μl |
| O.1 M DTT | 5.0 μl |
| whole RNA 250 ng/μl | 5.0 μl |
| T₁₂VV primers (V = A, C or G), 25 μM | 5.0 μl |
| 5 x RT buffer* | 10.0 μl |
| dH₂O | 20.3 μl |
| | 50.0 μl |

| | |
|---|---|
| *5 x RT buffer: | 250 mM Tris-HCl (pH 7.6) |
| | 375 mM KCl |
| | 15 mM MgCl₂ |

The whole RNA was denatured prior to the reaction at 70-80° C. for 5 minutes, then quenched on ice and fed to the reaction vessel. All of the other components were mixed at O° C. and added to the whole RNA. Ultimately all was coated with mineral oil and incubated in a water bath at 37° C. for 45-60 minutes. Finally, the reaction was stopped by inactivating the enzyme at 95° C. (5 minutes). Having terminated the reaction, the RT batches were frozen at −20° C. LIP to their use.

The resulting cDNA was subjected to a PCR method. To this end, 20 μl batches were made, each having 2 μl of the above reverse transcription batch as template ( 1/10 of the reaction volume, corresponding to the equivalent of 25-50 ng cDNA/PCR batch). The other components (cf. below) were prepared as “master mix” and then added. A PCR reaction batch was composed as follows:

| | |
|---|---|
| RT batch (prepared)l | 2.0 μl |
| Mix: | |
| 10 x PCR buffer* | 2.0 μl |
| 10-mer arbitrary primer 5 μM | 2.5 μl |
| T₁₂VV primers (V = A, C or G), 25 μM | 2.0 μl |
| dNTP mix (2.5 mM in toto) | 0.4 μl |
| 50 mM MgCl₂ | 0.7 μl |
| Taq DNA polymerase 20 U/μl | 0.2 μl |
| α-³²P-dCTP | 0.1 μl |
| dH₂O | 10.6 μl |
| | 20.0 μl |

| | |
|---|---|
| *10 x PCR buffer: | 200 mM Tris-HCl (pH 8.55) |
| | 160 mM (NH₄)₂SO₄ |
| | magnesium: optimum concentration |
| | is adjusted with 50 mM MgCl₂ |

The 10-mer arbitrary primer is e.g. “AGC CAG CGA A” (AP-1) or “GCA ATC GAT G” (AP-6). The reaction was carried out in a DNA thermocycler (Perkin-Elmer Gen-Amp 9600) with the following program steps:

| | | | |
|---|---|---|---|
| Program 1: | denaturation | 95° C., 3 minutes | 1 cycle |
| Program 2: | denaturation | 95° C., 15 seconds | a maximum |
| | primer annealing | 40° C., 2 minutes | of 30 |
| | primer extension | 72° C., 30 seconds | cycles |
| Program 3: | Primer extension | 72° C., 5 minutes | 1 cycle |

Having terminated the PCR method, the batches were applied to a denaturing 4.5-6% polyacrylamide gel. By comparing the cDNA bands from the early and late passages of the HPK-IA cells, those could be identified which were different, i.e. were represented in the late passages much more than in the early ones.

These cDNA bands were used for another amplification. To this end, they were cut out of the polyacrylamide gel and used in another PCR method. The PCR batch was composed as follows:

| | |
|---|---|
| 10 x PCR buffer | 5.0 μl |
| 10-mer arbitrary primer 5 μM | 5.0 μl |
| T₁₂VV primer (V = A, C or G), 25 μM | 5.0 μl |
| dNTP mix (25 mM in toto) | 1.2 μl |
| 50 mM MgCl₂ | 1.5 μl |
| Taq-DNA polymerase 20 U/μl | 1.0 μl |
| dH₂O | 31.5 μl |
| | 50.0 μl |

The PCR reaction was carried out under the same conditions and with the same program sequence as the first PCR reaction.

Having terminated the PCR reaction, the batches were separated on a 1% agarose gel, and the desired DNA bands were cut out of the gel. Thereafter, the DNA bands eluted from the agarose pieces by using what is called “GenElute” columns (SUPELCO company).

The resulting cDNA was cloned into the cloning vector pCRII using the “TA cloning kit” (INVITROGEN company). Resulting clones were sequenced by means of the “T7 sequencing kit” (PHARMACIA company). The cDNAs C4.8 and C21.7 according to the invention were obtained.

### EXAMPLE 2

### Comparative Studies Using cDNAs C4.8 and C21.7 According to the Invention

a) The whole RNA isolated according to Example 1 from early and late passages of HPK-IA cells was subjected to a denaturing 4.5-6% polyacrylamide gel electrophoresis. Thereafter, a conventional Northern blot was carried out, the RNA being transferred to “Gene Screen-Plus” nylon membranes. ³²P-labeled cDNAs C4.8 and C21.7 according to the invention were used for hybridization.

The results indicated that the cDNA according to the invention react much more strongly with the RNA from the late passages of the HPK-IA cells than was the case with the early passages.

b) Furthermore, RNA-RNA in situ hybridizations were made with freezing sections from cervical tissues, namely normal epithelial tissue, premalignant lesion and carcinoma to evaluate the condition of the tissue. RNA probes were used as hybridization probes, which were obtained from the cDNAs C4.8 and C21.7 according to the invention. To this end, the latter were linearized and RNA was synthesized by adding the corresponding RNA polymerase, preferably SP6 or T7, and ³²P-rUTP. The RNA-RNA in situ hybridization with the above tissue was carried out under stringent conditions, e.g. at 60° C.

It turned out that strong hybridization was only obtained in connection with the cervical carcinoma.

However, the hybridization was very low in the case of normal epithelial tissue.

The above data underline that the present invention is perfectly suited to detect potentially malignant cells in a cervical smear.

1.-14. (canceled)

15. A kit useful for diagnosis of cervical lesions and evaluation of the progression potential of cervical lesions in a testing sample, comprising a first, agent which determines presence or absence and/or level of a polypeptide characteristic of early or late passages of HPV immortalized cells within said testing sample, and having the amino acid sequence encoded by SEQ ID NO: 1 or SEQ ID NO: 2, wherein said first agent is selected from the group consisting of a polypeptide, an antibody or fragment thereof which specially bind to said polypeptide and a nucleic acid molecule which hybridizes to a nucleic acid molecule which encodes said polypeptide and a second agent which binds to said first agent for identifying binding of said first agent to the polypeptide.

16. An isolated antibody which specifically binds to a polypeptide characteristic of early of late passage of HPV immortalized cells, said polypeptide comprises the amino acid sequence encoded by SEQ ID NO: 2.

17. The isolated antibody according to claim 16, wherein said antibody is a polyclonal antibody.

18. The isolated antibody according to claim 16, wherein said antibody is a monoclonal antibody.

19. A fragment of the antibody of claim 16.

20. The fragment of an antibody according to claim 19, wherein said fragment is a Fab fragment.

21. The kit according to claim 16, wherein said first agent is a polyclonal antibody.

22. The kit according to claim 16, wherein said first agent is a monoclonal antibody.

23. The kit according to claim 16, wherein said first agent is a fragment of antibody.

24. The kit according to claim 23, wherein said fragment is a Fab′ fragment.

25. A method for detection and/or diagnosis of cervical lesion and evaluation of the progression potential of cervical lesions comprising obtaining a sample of a cervical lesion from a patient, determining the presence or absence and/or the level of a protein encoded by the nucleotide sequence of SEQ ID NO: 1 or the nucleotide sequence of SEQ ID NO: 2 from a sample of HPV cells taken from the cervical lesion, wherein the sample is selected from the group consisting of a) cervical smear; b) a sample containing cells of cervical origin; and c) a biopsy or microbiopsy comprising cervical cells or tissues.

26. The method according to claim 25, wherein the cervical lesions comprise precancerous stages, mild to severe dysplasias and/or microinvasive or invasive carcinomas.

27. The method according to claim 25, wherein the polypeptides characteristic for the early or late passages of HPV immortalized cells are obtainable by a method comprising;
isolating RNA from early and late passages of HPV immortalized cells;
comparing the mRNA present in the samples of early and late passages to each other;
determining characteristic differences in the levels of distinct mRNA molecules between the compared samples;
isolating the mRNA, which exhibit characteristic differences between early and late stages of HPV immortalized cells; and
obtaining the encoded polypeptide from the mRNA molecules detected.

28. The method according to claim 25, wherein the polypeptides characteristic for the early or late passages of HPV immortalized cells comprise fragments of the amino acid sequence depicted in SEQ ID NO: 1 of at least five consecutive amino acids.

29. The method according to claim 25, wherein the detection is carried out using an agent specifically binding to said polypeptides, wherein said agent is an antibody.

30. The method according to claim 25, wherein the detection is carried out by a method comprising at least one of the following:
western blot
ELISA
Immunoprecipitation
Immunofluorescence
Immunocyto-chemistry
